(19) United States
(12) Patent Application Publication
(10) Pub. No.: US 2010/0226992 A1
(43) Pub. Date: Sep. 9, 2010

(54) Pharmaceutical Composition for Delivery of Receptor Tyrosine Kinase Inhibiting (RTKi) Compounds to the Eye

(75) Inventor: Bhagwati P. Kabra, Euless, TX (US)

Correspondence Address: ALCON, IP LEGAL, TB4-8, 6201 SOUTH FREEWAY, FORT WORTH, TX, 76134, US

(73) Assignee: ALCON RESEARCH, LTD., Fort Worth, TX (US)

(21) Appl. No.: 12/716,385

(22) Filed: Mar. 3, 2010

Related U.S. Application Data
(60) Provisional application No. 61/156,984, filed on Mar. 3, 2009.

(51) Int. Cl. A61K 31/416; A61K 47/10; A61K 9/14; A61P 27/02

(52) U.S. Cl. 424/489; 514/772; 514/406; 977/773

(57) ABSTRACT

The present invention relates to development of efficacious pharmaceutical compositions in the form of intraocular suspensions comprising an anti-angiogenic compound in a therapeutically effective amount and a polyethylene glycol having a molecular weight of at least 2000, preferably at least 3000.

This application claims priority under 35 U.S.C. §119 to U.S. Provisional Patent Application No. 61/156,984 filed Mar. 3, 2009, the entire contents of which are incorporated herein by reference.

### BACKGROUND OF THE INVENTION

1. Field of the Invention

The present invention relates to unique compositions containing compounds with poor solubility and methods useful for treating pathological states that arise or are exacerbated by ocular angiogenesis, inflammation and vascular leakage such as AMD, DR, diabetic macular edema etc., and more specifically, to compositions containing agent is with anti-angiogenic, anti-inflammatory or anti-vascular permeability property for use in treating ocular disorders.

2. Description of the Related Art

Abnormal neovascularization or angiogenesis and enhanced vascular permeability are major causes for many ocular disorders including age-related macular degeneration (AMD), retinopathy of prematurity (ROP), ischemic retinal vein occlusions and diabetic retinopathy (DR). AMD and DR are among the most common cause of severe, irreversible vision loss. In these and related diseases, such as retinal vein occlusion, central vision loss is secondary to angiogenesis, the development of new blood vessels from pre-existing vasculature, and alterations in vascular permeability properties.

The angiogenic process is known by the activation of quiescent endothelial cells in pre-existing blood vessels. The normal retinal circulation is resistant to neovascular stimuli, and very little endothelial cell proliferation takes place in the retinal vessels. While there appear to be many stimuli for retinal neovascularization, including tissue hypoxia, inflammatory cell infiltration and penetration barrier breakdown, all increase the local concentration of cytokines (VEGF, PDGF, FGF, TNF, IGF etc.), integrins and proteinases resulting in the formation of new vessels, which then disrupt the organizational structure of the neural retina or break through the inner limiting membranes into the vitreous. Elevated cytokine levels can also disrupt endothelial cell tight junctions, leading to an increase in vascular leakage and retinal edema, and disruption of the organizational structure of the neural retina. Although VEGF is considered to be a major mediator of inflammatory cell infiltration, endothelial cell proliferation and vascular leakage, other growth factors, such as PDGF, FGF, TNF, and IGF etc., are involved in these processes. Therefore, growth factor inhibitors can play a significant role in inhibiting retinal damage and the associated loss of vision upon local delivery in the eye or via oral dosing.

There is no cure for the diseases caused by ocular neovascularization and enhanced vascular permeability. The current treatment procedures of AMD include laser photocoagulation and photodynamic therapy (PDT). The effects of photocoagulation on ocular neovascularization and increased vascular permeability are achieved only through the thermal destruction of retinal cells. PDT usually requires a slow infusion of the dye, followed by application of non-thermal laser-light. Treatment usually causes the abnormal vessels to temporarily stop or decrease their leaking. PDT treatment may have to be repeated every three months up to 3 to 4 times during the first year. Potential problems associated with PDT treatment include headaches, blurring, and decreased sharpness and gaps in vision and, in 1-4% of patients, a substantial decrease in vision with partial recovery in many patients. Moreover, immediately following PDT treatment, patients must avoid direct sunlight for 5 days to avoid sunburn. Recently, a recombinant humanized IgG monoclonal antibody fragment (ranibizumab) was approved in the US for treatment of patients with age-related macular degeneration. This drug is typically administered via intravitreal injection once a month.

Many compounds that may be considered potentially useful in treating ocular neovascularization and enhanced vascular permeability-related and other disorders, are poorly soluble in water. A poorly water soluble compound is a substance that is not soluble at a therapeutically effective concentration in an aqueous physiologically acceptable vehicle. Aqueous solubility is an important parameter in formulation development of a poorly water soluble compound. What is needed is a formulation that provides increased solubility of the compound while also providing sufficient bioavailability of the compound so as to maintain its therapeutic potential.

For many years, the pharmaceutical industry has been developing and discovering suspending agents useful in the preparation of pharmaceutical suspensions. Such suspensions are efficacious for the delivery of therapeutic agents and other uses. These suspensions can be used in a wide variety of applications such as parentral, topical, oral, is rectal or the like and, of particular importance to the present invention, ophthalmic, otic and nasal. Examples of such suspensions are described in U.S. Pat. Nos. 7,001,615; 6,359,016; 6,284,804; 6,139,794; 5,932,572; 5,461,081 and US Patent Publication Nos. 20060257487; 20060257486; 20060122277; 20030139382; 20020037877; all of which are incorporated herein by reference for all purposes.

Generally speaking, it is desirable for suspending agent to assist in maintaining a therapeutic agent suspended within a suspension (e.g., an aqueous suspension) for a relatively large amount of time without allowing the therapeutic agent to settle out of the suspension. However, many popular conventional suspending agents allow therapeutic agent to settle out of suspension rather quickly. Moreover, many popular suspending agents also allow the therapeutic agent to become relatively tightly packed within the suspension and may not allow the therapeutic agent to be easily re-suspended. As examples, non-ionic polymers such as hydroxypropyl cellulose and hydroxyethyl cellulose often allow the therapeutic agent to settle out of solution at undesirably high rates and allow the therapeutic agent to become tightly packed once settled.

In addition to the above, many conventionally used suspending agents have been found to be incompatible with ingredients that have recently become desirable within pharmaceutical compositions. As one example, in the ophthalmic industry, there has been a move toward antimicrobial agents such as polymeric quaternary ammonium compounds that exhibit relatively low toxicity, however, certain anionic suspending agents such as carbopol, xanthan gum and carboxymethyl cellulose can be incompatible with such antimicrobial agents under certain circumstances.

In view of the above, there is a need for a suspension and suspending agent that assist the therapeutic agent in remaining suspended in an aqueous or other environment and/or assist the therapeutic agent in resisting tight packing upon settling out of the suspension. Additionally or alternatively, there is a need for suspending agent that exhibits a high degree of compatibility with highly desirable low toxicity ingredients of the suspensions.

The present invention provides safe and effective suspensions for ocular administration of poorly soluble compounds for the treatment of ocular diseases caused by endothelial cell proliferation, vascular leakage, inflammation and angiogenesis.

### SUMMARY OF THE INVENTION

The present invention overcomes these and other drawbacks of the prior art by providing compositions in the form of intraocular suspensions for treating ocular diseases due to angiogenesis, enhanced endothelial cell proliferation, inflammation, or increased vascular permeability. Within one aspect of the present invention, a pharmaceutical composition is provided wherein a compound having poor water solubility is incorporated into an intraocular suspension containing polyethylene glycol (PEG) having a molecular weight of greater than 2000 as a suspending agent for delivery of the compound for use in vitreoretinal therapy, in treating angiogenesis-related ocular disorders, inhibiting neovascularization, controlling vascular permeability, treating inflammation, and improving vision. The suspension of the present invention will be administered to the eye of a patient suffering from an angiogenesis-related ocular disorder, neovascularization, vascular permeability, or inflammation, including diabetic retinopathy (DR), age-related macular degeneration (AMD), geographic atrophy, and retinal edema.

The bioavailability of the compounds for use in the compositions of the present invention is substantially enhanced via use of a higher molecular weight PEG (e.g., MW≧2000) in the composition. The compositions of the invention are suspensions, preferably for delivery through a needle (e.g., 27 gauge) thereby treating angiogenesis-related ocular disorders, inhibiting neovascularization, controlling vascular permeability, treating inflammation, and/or improving vision.

The concentration of the anti-angiogenic, anti-inflammatory, or anti-vascular permeability agent used in the aqueous compositions of the present invention varies depending on the ophthalmic diseases and the route of administration used, and any concentration may be employed as long as its effect is exhibited. Thus, although the concentration is not restricted, a concentration of 0.001% to 10 wt % is preferred. The concentration of PEG will vary depending on the concentration of active agent used in the formulation. Although the concentrations are not restricted, usually, the preferred concentration of the PEG in the intravitreal composition is from 10% to 55%, more preferred concentration is 15% to 45%, and most preferred concentration is 15% to 30%.

In another embodiment, posterior juxtascleral (PJ) and periocular (PO) formulations containing (a) an active agent, such as an anti-angiogenic compound, an anti-inflammatory compound, or an anti-vascular permeability agent; (b) a suitable amount of a high molecular weight PEG; (c) a suitable buffer; (d) optionally tonicity agents; (e) a suspending agent; and (f) a surfactant are provided.

In yet another embodiment, the present invention provides formulations for topical ocular dosing, which include (a) a therapeutically effective amount of an active agent, such as an anti-angiogenic agent, an anti-inflammatory compound, or an anti-vascular permeability agent; (b) a suspending agent; (c) a surfactant; (d) tonicity agent; (d) a high molecular weight PEG; and (e) a buffer.

A wide variety of molecules may be utilized within the scope of present invention, especially those molecules having very low solubility. As used herein, the term “poor solubility” is used to refer to a compound having solubility in water or vehicle that is well below its therapeutic window, typically less than 1000 μg/mL, preferably less than 500 μg/mL, and more preferably less than 200 μg/mL. It is desirable to have a concentration of soluble drug in the formulation such that the concentration of soluble drug in the vitreous is increased. The suspensions described herein will preferably contain at least 200 μg/mL, more preferably at least 500 μg/mL, and most preferably at least 1000 μg/mL for local ocular delivery to elicit desirable biological activities.

The compositions of the present invention are preferably administered to the eye of a patient suffering from an angiogenesis or enhanced vascular permeability related ocular, or a disorder characterized by neovascularization or vascular permeability, via posterior juxtascleral administration, intravitreal injection, or vitreoretinal therapy.

### DETAILED DESCRIPTION PREFERRED EMBODIMENTS

As noted above, the present invention provides compositions that contain an active agent having poor water solubility, for use in the treatment of ocular disorders caused by endothelial cell proliferation, enhanced vascular permeability, inflammation, or angiogenesis. The compositions of the invention are useful in treating disorders associated with microvascular pathology, increased vascular permeability and intraocular neovascularization, including diabetic retinopathy (DR), age-related macular degeneration, geographic atrophy (AMD) and retinal edema.

Briefly, within the context of the present invention, an active agent should be understood to be any molecule, either synthetic or naturally occurring, which acts to inhibit vascular growth, reduce vascular permeability, and/or decrease inflammation. In particular, the present invention provides compositions comprising an insoluble, or poorly is soluble, active agent in a therapeutically effective amount in an intraocular suspension containing high molecular weight PEG (i.e., MW≧2000) for ophthalmic use. As used herein, when referring to a PEG of a particular molecular weight, the term “PEG” will be followed by a number, indicating the molecular weight for that particular PEG. For example, PEG 400 refers to a PEG having a molecular weight of approximately 400. Of course, the skilled artisan will understand that a designation of PEG 400 will refer to a range of PEGs having molecular weights of about 400 and will encompass PEGs with molecular weights above or below 400 by anywhere from 1-50%

Polyethylene glycols (PEGs) are widely used in a variety of pharmaceutical formulations including parenteral, topical, ophthalmic, oral and rectal preparations. PEGs are stable, hydrophilic substances and are non-irritating to the skin.

The present invention is based, in part, upon the discovery that intraocular suspensions incorporating PEGs with higher molecular weights (i.e., MW≧2000) as a suspending agent provides a composition that can be delivered directly to the eye of a patient suffering from an ocular disorder via a needle.

A higher molecular weight PEG (MW≧2000) is preferred over low molecular weight PEG (e.g., PEG 400) because it keeps tonicity of the formulations within ophthalmically acceptable ranges, even at very high concentrations. This allows for injection of a higher volume of the composition (e.g., 100 μl) into the vitreous of the patient. Higher molecular weight PEGs will also remain in the vitreous for a longer period of time and may provide a higher concentration of the active agent over a longer period of time.

The use of PEG as a suspending agent in intraocular suspensions provides certain advantages over other types of compositions containing poorly soluble active agents. The high molecular weight PEG with concentrations >10% can increase density and viscosity of the suspensions. The density of PEG is about 1.08. Thus, a composition containing a high molecular weight PEG as a suspending agent may sink to the bottom of the vitreous when injected into the eye, whereas a composition based on a substance of lower density may remain at the site of injection or float within the vitreous.

The use of PEG as a suspending agent results in slow settling as well as loosely settled or flocculated sediment. This is in contrast to other non-ionic polymers, such as hydroxypropyl cellulose and hydroxyethyl cellulose, which generally increase only viscosity. As a result they slow down settling, but the settled sediment is tightly packed and is difficult to resuspend. The sediment in PEG-based suspensions is either flocculated or loosely packed, and therefore, is easy to resuspend.

Additional advantages related to the use of high molecular weight PEGs, as compared to conventional polymers include an increase in solubility of poorly soluble active agents and higher density. Increased solubility for poorly soluble active agents may allow for increased bioavailability of the active agent to the target tissues. Furthermore, high molecular weight PEG may stay in the vitreous for a longer period of time, thereby allowing sustained deliver of the active agent. The higher density of the suspension allows to the suspension to sink to the bottom of the vitreous, thereby avoiding obstruction of vision.

It is contemplated that any active agent that is poorly water soluble may be included in the compositions of the present invention. For example, anti-angiogenic agents, anti-inflammatory agents, or anti-vascular permeability agents are useful in the is compositions of the invention.

Preferred anti-angiogenic agents include, but are not limited to, receptor tyrosine kinase inhibitors (RTKi), in particular, those having a multi-targeted receptor profile such as that described in further detail herein; angiostatic cortisenes; MMP inhibitors; integrin inhibitors; PDGF antagonists; antiproliferatives; HIF-1 inhibitors; fibroblast growth factor inhibitors; epidermal growth factor inhibitors; TIMP inhibitors; insulin-like growth factor inhibitors; TNF inhibitors; antisense oligonucleotides; etc. and prodrugs of any of the aforementioned agents. The preferred anti-angiogenic agent for use in the present invention is a multi-targeted receptor tyrosine kinase inhibitor (RTKi). Most preferred are RTKi's with multi-target binding profiles, such as N-[4-(3-amino-1H-indazol-4-yl)phenyl]-N′-(2-fluoro-5-methylphenyl)urea, having the binding profile substantially similar to that listed in Table 1. Additional multi-targeted receptor tyrosine kinase inhibitors contemplated for use in the compositions of the present invention are described in U.S. Application Serial No. 2004/0235892, incorporated herein by reference. As used herein, the term “multi-targeted receptor tyrosine kinase inhibitor” refers to a compound having a receptor binding profile exhibiting selectivity for multiple receptors shown to be important in angiogenesis, such as the profile shown in Table 1, and described in co-pending U.S. application serial number 2006/0189608, incorporated herein by reference. More specifically, the preferred binding profile for the multi-targeted receptor tyrosine kinase inhibitor compounds for use in the compositions of the present invention is KDR (VEGFR2), Tie-2 and PDGFR.

TABLE 1
Kinase Selectivity Profile of a RTK Inhibitor

| KDR | FLT1 | FLT4 | PDGFR | CSF1R | KIT | FLT3 | TIE2 | FGFR | EGFR | SRC |
|---|---|---|---|---|---|---|---|---|---|---|
| 4 | 3 | 190 | 66 | 3 | 14 | 4 | 170 | >12,500 | >50,000 | >50,000 |

All data reported as IC50 values for kinase inhibition in cell-free enzymatic assays;
ND denotes no data.
Values determined @ 1 mM ATP.

Other agents which will be useful in the compositions and methods of the invention include anti-VEGF antibody (i.e., bevacizumab or ranibizumab); VEGF trap; siRNA molecules, or a mixture thereof, targeting at least two of the tyrosine kinase receptors having IC₅₀ values of less than 200 nM in Table 1; glucocorticoids (i.e., dexamethasone, fluoromethalone, medrysone, betamethasone, triamcinolone, triamcinolone acetonide, prednisone, prednisolone, hydrocortisone, rimexolone, and pharmaceutically acceptable salts thereof, prednicarbate, deflazacort, halomethasone, tixocortol, prednylidene (21-diethylaminoacetate), prednival, paramethasone, methylprednisolone, meprednisone, mazipredone, isoflupredone, halopredone acetate, halcinonide, formocortal, flurandrenolide, fluprednisolone, fluprednidine acetate, fluperolone acetate, fluocortolone, fluocortin butyl, fluocinonide, fluocinolone acetonide, flunisolide, flumethasone, fludrocortisone, fluclorinide, enoxolone, difluprednate, diflucortolone, diflorasone diacetate, desoximetasone (desoxymethasone), desonide, descinolone, cortivazol, corticosterone, cortisone, cloprednol, clocortolone, clobetasone, clobetasol, chloroprednisone, cafestol, budesonide, beclomethasone, amcinonide, allopregnane acetonide, alclometasone, 21-acetoxypregnenolone, tralonide, diflorasone acetate, deacylcortivazol, RU-26988, budesonide, and deacylcortivazol oxetanone); Naphthohydroquinone antibiotics (i.e., Rifamycin); and NSAIDs (i.e., nepafenac, amfenac). The RTKi compound N-[4-(3-amino-1H-indazol-4-yl)phenyl]-N′-(2-fluoro-5-methylphenyl)urea has extremely poor solubility in phosphate buffer, pH 7.2 (0.00059 mg/mL), and is contemplated to be useful in the suspensions of the present invention.

The volume mean particle size (diameter) of all suspended or suspendable therapeutic agent in the suspension is typically at least 0.1 nm, more typically at least 1.0 μm and even more typically at least 2.0 μm. The volume mean diameter particle size of is all suspended or suspendable therapeutic agent in the suspension is typically no greater than 20 μm, more typically no greater than 10 μm and even more typically no greater than 5 μm. In certain embodiments, the mean diameter particle size of all suspended or suspendable therapeutic agent in the suspension is between about 1000 nm and 2000 nm. In preferred aspects of the invention, the mean diameter particle size of the active agent will be from about 1150 nm to about 1400 nm, more preferably about 1225 nm to about 1250 nm. In one preferred embodiment, the mean diameter particle size of the active agent in the suspension is about 1237 nm. In other preferred aspects of the invention, the mean diameter particle size of the active agent will be from about 1500 nm to about 1750 nm, more preferably about 1635 nm to about 1660 nm. In another preferred embodiment, the mean diameter particle size of the active agent in the suspension is about 1648 nm.

It is contemplated that virtually any PEG with a molecular weight greater than 2000 can be used in the compositions and methods of the invention. Preferred PEGs for use in the compositions and methods of the invention include PEG 3000, PEG 4000, PEG 6000, PEG 8000, PEG 14000 and PEG 20000. It is further contemplated that mixtures of higher molecular PEGs, for example mixtures of PEG 3000 and PEG 20000 or mixtures of PEG 6000 and PEG 20000, may be utilized in the compositions and methods of the invention.

The formulations of the present invention provide a number of advantages over conventional formulations. One advantage of the present invention is that PEGs can successfully solubilize poorly soluble compounds, allowing the preparation of an efficacious opthalmologically acceptable intravitreal, PJ and/or periocular formulation for local ocular delivery. Additionally, bioavailability of the drug can be modulated by controlling the molecular weight of the PEG used in the formulation. Furthermore, the preparation can be injected using a 27 or 30 gauge needle. Another advantage of the compositions of the present invention is that toxicity of the active compound can be reduced or suitably modulated.

The present inventors have discovered that use of higher molecular weight PEGs as a suspending agent to solubilize and deliver highly insoluble anti-angiogenic active compounds provides an efficacious ophthalmic formulation. Additionally, the active agent may be delivered to the ocular tissues of a patient treated with the ophthalmic suspensions described herein for a longer period of time than active agents currently used for treatment of such disorders. For example, the ophthalmic suspensions of the present invention are contemplated to deliver active agent to the ocular tissues of a patient for at least two months. In other embodiments of the present invention, the active agent will be delivered to the ocular tissues of the patient for at least three months or for at least four months. Another advantage of the suspensions of the present invention is that the particles of the active tend to form loose floccules, thereby resulting in a high degree of flocculation. The high degree of flocculation of the suspensions of the present invention ensures that they redisperse or resuspend easily upon gentle shaking.

In certain preferred embodiments, the formulation of the invention will further comprise a suitable viscosity agent, such as hydroxypropyl methylcellulose, hydroxyethyl cellulose, polyvinylpyrrolilidone, carboxymethyl cellulose, polyvinyl alcohol, sodium chondrointin sulfate, sodium hyaluronate etc. as a dispersant, if necessary. A nonionic surfactant such as polysorbate 80, polysorbate 20, tyloxapol, Cremophor, HCO 40 etc. can be used. The ophthalmic preparation according to the present invention may contain a suitable buffering system, such as phosphate, citrate, borate, tris, etc., and pH regulators such as sodium hydroxide and hydrochloric acid may also be used in the formulations of the inventions. Sodium chloride or other tonicity agents may be used to adjust tonicity, if necessary.

The suspensions of the present invention will typically have a pH in the range of 4 to 9, preferably 5.5 to 8.5, and most preferably 5.5 to 8.0. Particularly desired pH ranges are 6.0 to 7.8 and more specifically 6.4 to 7.6. The compositions will have an osmolality of 200 to 400 or 450 milliosmoles per kilogram (mOsm/kg), more preferably 240 to 360 mOsm/kg).

The specific dose level of the active agent for any particular human or animal depends upon a variety of factors, including the activity of the active compound used, the age, body weight, general health, time of administration, route of administration, and the severity of the pathologic condition undergoing therapy.

The formulations described herein may be delivered via intravitreal injection, via posterior juxtascleral, and periocular routes. In preferred embodiments of the present invention, the amount of active agent, or poorly water soluble agent, in the suspension will be from about 0.001% to 20% for intravitreal administration. More preferably from 0.05% to 18% and most preferably from 0.1% to 10%.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Suspension Containing PEG 14000

A solution containing sodium chloride, dibasic sodium phosphate dodecahydrate and PEG 14000 was heated. The compound N-[4-(3-amino-1H-indazol-4-yl) phenyl]-N′-(2-fluoro-5-methylphenyl)urea was added to it and dissolved. Upon cooling, the solution formed a milky suspension. After five weeks of observation, the suspension had not settled.

| Ingredients | W/W % |
|---|---|
| Active Agent | 1 |
| Sodium Chloride | 0.7 |
| Dibasic sodium phosphate, | 0.1 |
| dodecahydrate | |
| Polyethylene glycol 14000 | 48 |
| Water for injection | QS 100% |

### Example 2

### Suspension Containing PEG 20000

The following formulation was prepared using standard procedures. Active agent was milled in the presence of Polysorbate 80. The resulting slurry was added to a solution containing the other ingredients. The formulation had not formed a sediment after 10 days and can be easily resuspended.

| Ingredients | W/W % |
|---|---|
| Active Agent | 1 |
| Polysorbate 80 | 0.05 |
| Sodium Chloride | 0.7 |
| Dibasic sodium phosphate, | 0.05 |
| dodecahydrate | |
| Monobasic sodium phosphate, | 0.005 |
| dehydrate | |
| Polyethylene glycol 20000 | 23 |
| Sodium hydroxide | Adjust to pH 7.4 |
| Hydrochloric acid | Adjust to pH 7.4 |
| Water for injection | QS 100% |

### Example 3

### Suspension Containing a Mixture of PEG 6000 and PEG 20000

The following formulation was prepared using standard procedures. Active agent was milled in the presence of Polysorbate 80. The resulting slurry was added to a solution containing the other ingredients. The formulation had not formed a sediment after 10 days and can be easily resuspended.

| Ingredients | W/W % |
|---|---|
| Active Agent | 0.6 |
| Polysorbate 80 | 0.03 |
| Sodium Chloride | 0.4 |
| Dibasic sodium phosphate, | 0.05 |
| dodecahydrate | |
| Monobasic sodium phosphate, | 0.005 |
| dehydrate | |
| Polyethylene glycol 6000 | 35 |
| Polyethylene glycol 20000 | 10 |
| Sodium hydroxide | Adjust to pH 7.4 |
| Hydrochloric acid | Adjust to pH 7.4 |
| Water for injection | QS 100% |

### Examples 4 and 5

The compositions of two non-aqueous solution of RTKi in low molecular weight PEG are provided below.

Examples

| | 4 | 5 |
|---|---|---|
| Ingredients | W/V % | W/V % |
| RTKi | 3 | 7.5 |
| PEG 400 | 97 | 92.5 |

A pharmacokinetic study was performed in FIX rabbits by giving a 20 μl an injection of non-aqueous PEG based solutions to inferotemporal quadrant of the vitreous. The levels of RTKi observed in the central retina were determined by LC/MS/MS analysis. These levels are provided below.

| Examples | 4 | 5 |
|---|---|---|
| Injection Volume (μl) | 20 | 20 |
| Dose (μg) | 600 | 1500 |
| RTKi concentration (μM) in Retina at Day 2 | 4.6 | 5.0 |
| RTKi concentration (μM) in Retina at Day 14 | 1.7 | 1.5 |
| RTKi concentration (μM) in Retina at Day 56 | 0.34 | 0.86 |

### Examples 6 and 7

The compositions of a slightly higher molecular weight based PEG suspensions are to provided below. The particle size of RTKi was reduced by wet milling of RTKi in the presence of a surfactant using zirconium beads. RTKi slurry was combined with aqueous solutions of high molecular weight PEG and sodium chloride and phosphate buffer.

TABLE 3

| | 6 | 7 |
|---|---|---|
| Ingredients | W/V % | W/V % |
| RTKi | 1.4 | 1.4 |
| Polysorbate 80 | — | 0.14 |
| Tyloxapol | 0.14 | — |
| PEG 3000 | 15 | 15 |
| Sodium Dihydrogen | 0.025 | 0.025 |
| Phosphate, Dihydrate | | |
| Dibasic Sodium Phosphate, | 0.25 | 0.25 |
| Dodecahydrate | | |
| Sodium Chloride | 0.4 | 0.4 |
| Sodium Hydroxide or | Adjust | Adjust |
| Hydrochloric Acid | pH 7.4 | pH 7.4 |
| WFI | Qs | Qs |

The viscosity measured using a Brookfield viscometer. The mean volume average particle size was measured using Microtrac. These results are provided below. These suspensions were highly flocculated. The density of compositions 4 and 5 was found to be approximately 1.02.

| Examples | 6 | 7 |
|---|---|---|
| Mean Vol. Particle Size | 400 | 670 |
| (Microtrac), nm | | |
| Viscosity, cps | 5 | 5 |

A pharmacokinetic study was performed in FIX rabbits by giving a 100 μl an injection of the suspension to inferotemporal quadrant of the vitreous. The levels of RTKi to observed in the central retina and vitreous were determined by LC/MS/MS analysis. These levels are provided below. The central retina levels from examples 6 and 7 are much higher than those of low molecular PEG based non-aqueous solutions from examples 4 and 5. Though, the central retina levels from examples 6 and 7 are very high at day 2 and 14, they tend to drop significantly at day 56.

| Composition | 6 | 7 |
|---|---|---|
| Injection Volume (μl) | 100 | 100 |
| Dose (μg) | 1400 | 1400 |
| RTKi concentration (μM) in | 74 | 86 |
| Retina at Day 2 | | |
| RTKi concentration (μM) in | 69 | 151 |
| Retina at Day 14 | | |
| RTKi concentration (μM) in | 8 | 38 |
| Retina at Day 56 | | |
| Total RTKi amount(μg) in | 1010 | 932 |
| Vitreous at Day 14 | | |
| Total RTKi amount(μg) in | 814 | 392 |
| Vitreous at Day 56 | | |

### Examples 8, 9 and 10

The compositions of these higher molecular weight based PEG suspensions is provided below. The particle size of RTKi was reduced by wet milling of RTKi in the presence of a surfactant using zirconium beads. RTKi slurry was combined with aqueous solutions of high molecular weight PEG and sodium chloride and phosphate buffer.

| Example | 8 | 9 | 10 |
|---|---|---|---|
| RTKi | 1 | 1 | 1 |
| Polysorbate 80 | 0.1 | 0.1 | 0.1 |
| PEG 3000 | 15 | | |
| PEG 20000 | — | 25 | 25 |
| Sodium Dihydrogen | 0.025 | 0.025 | 0.025 |
| Phosphate, Dihydrate | | | |
| Dibasic Sodium Phosphate, | 0.25 | 0.25 | 0.25 |
| Dodecahydrate | | | |
| Sodium Chloride | 0.4 | 0.4 | 0.4 |
| Sodium Hydroxide or | Adjust | Adjust | Adjust |
| Hydrochloric Acid | pH 7.4 | pH 7.4 | pH 7.4 |
| WFI | Qs | Qs | Qs |

The viscosity measured using a Brookfield viscometer. The mean volume average to particle size was measured using Microtrac. These results are provided below. These suspensions were highly flocculated.

| Example | 8 | 9 | 10 |
|---|---|---|---|
| Mean Vol. Particle Size | 1201 | 1237 | 1648 |
| (Microtrac), nm | | | |
| Viscosity, cps | 5 | 150 | 150 |

A pharmacokinetic study was performed in FIX rabbits by giving a 100 μl an injection of the suspension to inferotemporal quadrant of the vitreous. The levels of RTKi observed in the central retina and vitreous were determined by LC/MS/MS analysis. These levels are provided below. The results show that for example 8, the central retina level tend to drop significantly at day 56 compared to day 2 and 14. However, the central retina levels Day 56 of examples 9 and 10 at which uses significantly higher molecular weight PEG 20000 are similar to those observed at Day 2 and 14. This suggests 20000 molecular weight PEG facilitate higher levels of the drug in central retina even at day 56 compare to 3000 molecular weight PEG.

| Example | 8 | 9 | 10 |
|---|---|---|---|
| Injection Volume (μl) | 100 | 100 | 100 |
| Dose (μg) | 1000 | 1000 | 1000 |
| RTKi concentration (μM) in | 19 | 24 | 11 |
| Retina at Day 2 | | | |
| RTKi concentration (μM) in | 11 | 15 | 10 |
| Retina at Day 14 | | | |
| RTKi concentration (μM) in | 9 | 17 | 9 |
| Retina at Day 35 | | | |
| RTKi concentration (μM) in | 3 | 16 | 7 |
| Retina at Day 56 | | | |
| Total RTKi amount(μg) in | 893 | 711 | 955 |
| Vitreous at Day 2 | | | |
| Total RTKi amount(μg) in | 776 | 656 | 814 |
| Vitreous at Day 14 | | | |
| Total RTKi amount(μg) in | 668 | 418 | 574 |
| Vitreous at Day 35 | | | |
| Total RTKi amount(μg) in | 540 | 312 | 377 |
| Vitreous at Day 56 | | | |

All of the compositions and/or methods disclosed and claimed herein can be made and executed without undue experimentation in light of the present disclosure. While the compositions and methods of this invention have been described in terms of preferred to embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and structurally related may be substituted for the agents described herein to achieve similar results. All such substitutions and modifications apparent to those skilled in the art are deemed to be within the spirit, scope and concept of the invention as defined by the appended claims.

### REFERENCES

All references cited herein, to the extent that they provide exemplary procedural or other details supplementary to those set forth herein, are specifically incorporated herein by reference.

We claim:

1. An ophthalmic suspension for treating ocular neovascularization, said composition comprising:
a poorly water soluble active agent in an amount of from 0.01% to 20%, and a polyethylene glycol having a molecular weight of at least 2000 in an amount from 5% to 50%.

2. The suspension of claim 1, wherein the active agent is selected from the group consisting of anti-angiogenic agents, anti-inflammatory agents, and anti-vascular permeability agents.

3. The suspension of claim 2, wherein the active agent is an anti-angiogenic agent.

4. The suspension of claim 3, wherein the anti-angiogenic agent is a multi-targeted receptor tyrosine kinase (RTK) inhibitor.

5. The suspension of claim 4, wherein the RTK inhibitor is N-[4-(3-amino-1H-indazol-4-yl)phenyl]-N′-(2-fluoro-5-methylphenyl)urea.

6. The suspension of claim 2, wherein the said concentration of the anti-angiogenic agent is from 0.001% to 10%.

7. The suspension of claim 6, wherein the PEG has a molecular weight of at least 3000.

8. The suspension of claim 7, wherein the concentration of PEG in the formulation is from 10% to 50%.

9. The suspension of claim 8, further comprising a nonionic surfactant selected from the group consisting of polysorbate 80, polysorbate 20, tyloxapol, Cremophor, and HCO 40.

10. The suspension of claim 7, wherein the PEG is selected from the group consisting of PEG 3000, PEG 20000, and a mixture of PEG 3000 and PEG 20000.

11. The suspension of claim 10, further comprising a nonionic surfactant selected from the group consisting of polysorbate 80, polysorbate 20, tyloxapol, Cremophor, and HCO 40.

12. The suspension of claim 9, comprising 1% of the active agent N-[4-(3-amino-1H-indazol-4-yl)phenyl]-N′-(2-fluoro-5-methylphenyl)urea and 48% of PEG 14000.

13. The suspension of claim 7, wherein the molecular weight of the PEG is 20000.

14. The suspension of claim 13, further comprising PEG 6000.

15. The suspension of claim 14, wherein the concentration of the RTK inhibitor in the suspension is 0.6% (w/w), the concentration of PEG 6000 is 35% (w/w) and the concentration of PEG 20000 is 10% (w/w).

16. The suspension of claim 13, wherein the concentration of the RTK inhibitor in the is suspension is 1% (w/w) and the concentration of the PEG 20000 is 23% (w/w).

17. The suspension of claim 10, comprising
1.4% (w/v) active agent; and
15% (w/v) PEG 3000.

18. The suspension of claim 17, further comprising 0.14% (w/v) Polysorbate 80.

19. The suspension of claim 17, further comprising 0.14% (w/v) Tyloxapol.

20. The suspension of claim 11, comprising
1% (w/v) active agent;
15% (w/v) PEG 3000; and
0.1% (w/v) polysorbate 80.

21. The suspension of claim 11, comprising
1% (w/v) active agent;
25% (w/v) PEG 20000; and
0.1% (w/v) polysorbate 80.

22. The suspension of claim 21, wherein the particle size of the active agent is from about 1000 nm to about 2000 nm.

23. The suspension of claim 22, wherein the particle size of active agent is from about 1150 nm to about 1400 nm.

24. The suspension of claim 23, wherein the particle size of active agent is about 1237 nm.

25. The suspension of claim 22, wherein the particle size of the active agent is from about 1500 nm to about 1750 nm.

26. The suspension of claim 25, wherein the particle size of the active agent is about 1648 nm.

27. An ophthalmic suspension for intravitreal injection for the treatment of ocular disorders associated with neovascularization, said suspension comprising from 0.1 to 20% of a multi-targeted receptor tyrosine kinase inhibitor and a polyethylene glycol having a molecular weight of at least 4000.

28. An ophthalmic suspension for posterior juxtascleral or periocular injection for the treatment of ocular disorders associated with neovascularization, said composition comprising from 0.5 to 20% of a multi-targeted receptor tyrosine kinase inhibitor and a polyethylene glycol having a molecular weight of at least 4000.

29. The suspension of claim 27, wherein the RTK inhibitor is N-[4-(3-amino-1H-indazol-4-yl)phenyl]-N′-(2-fluoro-5-methylphenyl)urea and the PEG is PEG 20000.

30. The suspension of claim 28, wherein the RTK inhibitor is N-[4-(3-amino-1H-indazol-4-yl)phenyl]-N′-(2-fluoro-5-methylphenyl)urea and the PEG is PEG 20000.

31. A method for treating an ocular disorder associated with microvascular pathology, increased vascular permeability or intraocular neovascularization, said method comprising is administering to the eye of a patient suffering from said ocular disorder an ophthalmic suspension of claim 1.

32. The method of claim 31, wherein said ocular disorder is selected from the group consisting of diabetic retinopathy, age-related macular degeneration, macular edema, uveitis, and geographic atrophy.

33. The method of claim 32, wherein the composition is the composition of claim 15.

34. The method of claim 32, wherein the composition is the composition of claim 16.

35. The method of claim 32, wherein the composition is the composition of claim 17.

36. The method of claim 32, wherein the composition is the composition of claim 18.

37. The method of claim 32, wherein the composition is the composition of claim 19.

38. The method of claim 32, wherein the composition is the composition of claim 20.

39. The method of claim 32, wherein the composition is the composition of claim 21.

40. The method of claim 32, wherein the composition is the composition of claim 22.

41. The method of claim 32, wherein the duration of delivery of the active agent to the ocular tissues of the patient after injection of the suspension is at least two months.
